(19) United States
(12) Patent Application Publication
(10) Pub. No.: US 2023/0001261 A1
(43) Pub. Date: Jan. 5, 2023

(54) SMART MAT

(71) Applicant: OMOLLE CO., LTD, Seoul (KR)

(72) Inventors: Min Ki KANG, Seongnam-si (KR); Sung Ho YOO, Seoul (KR); Seong Jin HONG, Bucheon-si (KR); Tae Jun PARK, Seongnam-si (KR); Gi Yeon NAM, Seoul (KR)

(21) Appl. No.: 17/707,449

(22) Filed: Mar. 29, 2022

(30) Foreign Application Priority Data
Jul. 1, 2021 (KR) PCT/KR2021/008379
Jul. 1, 2021 (KR) PCT/KR2021/008388
Mar. 28, 2022 (KR) 10-2022-0038265

(51) Int. Cl. A63B 21/00; A63B 71/06

(52) U.S. Cl. CPC A63B 21/4037; A63B 71/06; A63B 2071/0675

(57) ABSTRACT

A key input device applicable to a smart mat is provided, where the key input device includes a first layer in which a plurality of row contacts are formed, a second layer in which a plurality of column contacts are formed, an insulating layer disposed between the first and second layers to form an insulating region and a current carrying region, and a processor configured to detect that at least one of a plurality of key switches formed by the plurality of row contacts and the plurality of column contacts is pressed.

### CROSS-REFERENCE TO RELATED APPLICATIONS

This application claims priority to Korean Patent Application No. 10-2022-0038265, filed on Mar. 28, 2022, International Application No. PCT/KR2021/008388, filed on Jul. 1, 2021, and International Application No. PCT/KR2021/008379, filed on Jul. 1, 2021, the contents of which are incorporated by reference in their entirety.

### BACKGROUND

### 1. Field of the Invention

The present disclosure relates to a smart mat, and more specifically, to a smart mat for improving recognition accuracy when applied to a large area such as a mat.

### 2. Description of Related Art

A key input device used as an input device for a PC or a notebook computer is a typical key input device. In the key input device, a plurality of keys are arranged, and a detection result is transmitted to the PC or the notebook computer by detecting that the key is pressed.

For effective key input detection, it is common to use a detecting structure in the form of a key matrix.

When using this key matrix structure, a user's touch or pressure applied to a plate-shaped mat installed on a floor or wall may be detected. As an example, a key input device is provided in a layered form in which the key input device is stacked inside a training mat installed on the floor, so that user's motion on the mat may be detected.

However, the existing key matrix structure is optimized when a plurality of physical keys are provided in a narrow region such as a keyboard, and thus there is a problem in that recognition accuracy is lowered when applied to a large region such as a mat.

In addition, the existing key matrix structure has a problem in that misrecognition occurs due to ghosting when there are key inputs at three or more points.

Therefore, research on the key matrix structure that minimizes misrecognition and enables accurate recognition even if the key matrix structure is applied to a wider region than in the case of the existing key matrix structure.

### SUMMARY

The present disclosure provides a smart mat capable of inputting a key with high accuracy without switching using a key having a physical form.

The present disclosure also provides a smart mat that detects a user's contact or pressurization on a large region such as a mat.

The present disclosure also provides a smart mat that minimizes misrecognition such as ghosting even when a plurality of points are simultaneously pressed.

Embodiments to be implemented in the present disclosure are not limited to the embodiments mentioned above, and other embodiments not mentioned will be clearly understood by those of ordinary skill in the art to which the present disclosure belongs from the following description

In accordance with an exemplary embodiment of the present invention, there is provided a smart mat including: a first layer in which a plurality of row contacts are formed; a second layer in which a plurality of column contacts are formed; an insulating layer disposed between the first and second layers to form an insulating region and a current carrying region, and a processor configured to detect that at least one of a plurality of key switches formed by the plurality of row contacts and the plurality of column contacts is pressed.

A key region may be formed in a region where the plurality of row and column contacts cross each other.

The current carrying region may be formed to correspond to the key region.

The plurality of row contacts may include a plurality of row contact units connected to each other, and the plurality of column contacts may include a plurality of column contact units connected to each other.

The key region may be formed at a point where the plurality of row contact units and column contact units cross each other.

The insulating region may be formed to insulate a region where the row contact unit and the column contact unit cross along a line.

The current carrying region may be formed such that the current is allowed to be carried in a region where the row contact unit and the column contact unit cross each other at a point.

Additional scope of applicability of the present disclosure will become apparent from the following detailed description. However, since various alterations and modifications within the technical spirit and scope of the present disclosure can be clearly understood by those skilled in the art to which the present disclosure belongs, it should be understood that the specific embodiments described in the detailed description are given by way of example only.

### BRIEF DESCRIPTION OF THE DRAWINGS

FIG. 1 illustrates a conceptual diagram of an interactive fitness system 100 in accordance with exemplary embodiment of the present invention.

FIG. 2 is a conceptual diagram for describing an interactive action in accordance with an exemplary embodiment of the present invention.

FIG. 3 illustrates a training screen of a user terminal 130 for implementing the interactive action in accordance with another exemplary embodiment of the present invention.

FIG. 4 illustrates a block diagram of a smart mat 110 in accordance with still another exemplary embodiment of the present invention.

FIG. 5 is a diagram illustrating a general key detection method based on a key matrix structure.

FIG. 6 illustrates a stacked structure of a key input device 150 in accordance with still another exemplary embodiment of the present invention.

FIG. 7 illustrates a block diagram of the key input device 150 in accordance with still another exemplary embodiment of the present invention.

FIG. 8 illustrates a conceptual diagram in which a plurality of row and column contacts 611 and 612 are formed in accordance with still another exemplary embodiment of the present invention.

FIG. 9 illustrates key regions A1 to E4 formed by the plurality of row and column contacts 611 and 612 in accordance with still another exemplary embodiment of the present invention.

FIG. 10 is a diagram illustrating a first contact pattern 1001-1 in accordance with a first embodiment of the present invention.

FIG. 11 is an enlarged diagram of a part of the first contact pattern 1001-1 of FIG. 10.

FIG. 12 is a diagram illustrating a second contact pattern 1001-2 in accordance with a second embodiment of the present invention.

FIG. 13 is an enlarged diagram of a part of the second contact pattern 1001-2 of FIG. 12.

FIG. 14 is a diagram illustrating a third contact pattern 1001-3 in accordance with a third embodiment of the present invention.

FIG. 15 is a diagram illustrating a shape of a row contact unit 1401 in accordance with still another exemplary embodiment of the present invention.

FIG. 16 illustrates a conceptual diagram for dividing an entire area of the smart mat 110 in accordance with still another exemplary embodiment of the present invention.

FIG. 17 illustrates a spacer dot pattern 1701 formed in row/column contact units 1401 and 1402 in accordance with still another exemplary embodiment of the present invention.

FIG. 18 illustrates an insulating structure of first and second layers 601 and 603 in accordance with still another exemplary embodiment of the present invention.

FIG. 19 illustrates an adhesive structure of the first and second layers 601 and 603 in accordance with still another exemplary embodiment of the present invention.

FIG. 20 is a diagram illustrating a row dead space 2001 for a plurality of row contacts formed on the first layer 601 in accordance with still another exemplary embodiment of the present invention.

FIG. 21 is a diagram illustrating a column dead space 2002 for a plurality of column contacts formed on the second layer 603 in accordance with still another exemplary embodiment of the present invention.

FIG. 22 is a conceptual diagram for describing a shape in which electrode lines cross each other.

FIG. 23 is a diagram illustrating an example of a shape in which row/column contact units 1401 and 1402 cross each other in accordance with still another exemplary embodiment of the present invention.

FIG. 24 illustrates a first example of a crossing structure of the row/column contact units 1401 and 1402 in accordance with still another exemplary embodiment of the present invention.

FIG. 25 illustrates a second example of the crossing structure of the row/column contact units 1401 and 1402 in accordance with still another exemplary embodiment of the present invention.

FIG. 26 illustrates a third example of the crossing structure of the row/column contact units 1401 and 1402 in accordance with still another exemplary embodiment of the present invention.

FIG. 27 illustrates a fourth example of the crossing structure of the row/column contact units 1401 and 1402 in accordance with still another exemplary embodiment of the present invention.

FIG. 28 illustrates a fifth example of the crossing structure of the row/column contact units 1401 and 1402 in accordance with still another exemplary embodiment of the present invention.

FIG. 29 illustrates a form of a current carrying region 2801 according to the fifth example in more detail.

### DETAILED DESCRIPTION OF EMBODIMENTS

Hereinafter, the embodiments disclosed in the present specification will be described in detail with reference to the accompanying drawings, but the same or similar components are assigned the same reference numerals regardless of reference symbols, and redundant descriptions thereof will be omitted. The suffixes “module” and “unit” for components used in the following description are given or mixed in consideration of only the ease of writing the specification, and do not have distinct meanings or roles by themselves. In addition, in describing the embodiments disclosed in the present specification, if it is determined that a detailed description of a related known technology may obscure the gist of the embodiments disclosed in the present specification, the detailed description thereof will be omitted. In addition, the accompanying drawings are only for easy understanding of the embodiments disclosed in the present specification, and the technical ideas disclosed in the present specification are not limited by the accompanying drawings, and it should be understood that the accompanying drawings include all alterations, equivalents, and substitutions included in the spirit and technical scope of the present invention.

Terms including ordinal numbers such as first, second, etc. may be used to describe various components, but the components are not limited by the terms. The above terms are used only for the purpose of distinguishing one component from other components.

When it is mentioned that a certain component is “coupled” or “connected” to another component, it should be understood that the component may be directly coupled to or connected to the other component, but other component may exist therebetween. In contrast, when it is mentioned that a certain element is “directly coupled” or “directly connected” to another element, it should be understood that no other element exists therebetween.

The singular expression includes the plural expression unless the context clearly dictates otherwise.

In the present application, terms such as “comprise” or “have” are intended to designate that a feature, number, step, action, component, part, or combination thereof described in the specification is present, and it should be understood that the terms do not preclude the possibility of addition or existence of one or more other features or numbers, steps, actions, components, parts, or combinations thereof.

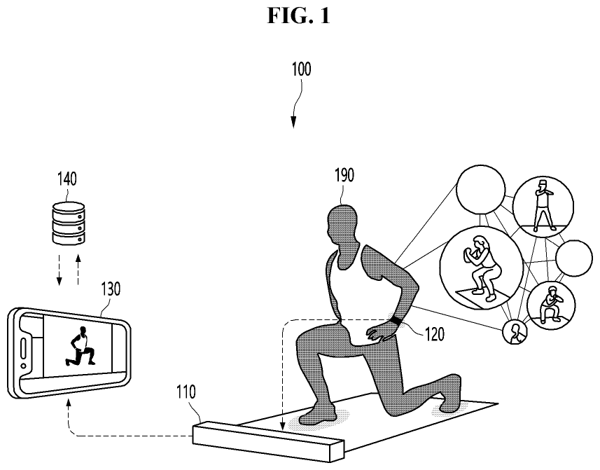

FIG. 1 illustrates a conceptual diagram of the interactive fitness system 100 in accordance with exemplary embodiment of the present invention.

The interactive fitness system 100 according to an embodiment of the present disclosure can be configured to include a smart mat 110, a wearable terminal 120, a user terminal 130, and a server 140. The components illustrated in FIG. 1 are not essential in implementing the interactive fitness system 100, and thus the interactive fitness system 100 described in this specification can include more or fewer components than those listed above.

The smart mat 110 is provided to acquire contact data, which is data obtained by detecting contact with at least a part of a body of a user 190. As a representative example, the smart mat 110 can be generally provided in the form of a yoga mat used at home or a fitness center and detect that a part of the body, such as a sole, a knee, a hand, or a hip comes into contact with the smart mat 110 when the user 190 performs various actions on the smart mat 110. It may be obvious that the smart mat 110 can detect a plurality of contact points at the same time.

The wearable terminal 120 is configured to be worn on a part of a body of the user 190 and to acquire motion data, which is data obtained by detecting motion of the part of the body. The wearable terminal 120 according to an embodiment of the present disclosure can be provided in the form of a watch or a band worn on the wrist of the user 190. As an example, the wearable terminal 120 is a smart watch (e.g., a Galaxy watch or an Apple watch), and embodiments of the present disclosure may be implemented through sensors provided in the smart watch.

Hereinafter, the contact data and the motion data are collectively referred to as action data.

As illustrated in the drawing, the motion data detected by the wearable terminal 120 may be transmitted to the user terminal 130 through the smart mat 110, but may not necessarily be limited thereto. That is, the wearable terminal 120 may exchange data with the smart mat 110 as illustrated in the drawing, but may exchange data with the user terminal 130, or the wearable terminal 120 may be directly connected to an online/offline network to exchange data with the server 140.

The user terminal 130 is a configuration unit for receiving data for analyzing behavior of the user 190 from the smart mat 110 and the wearable terminal 120 and transmitting the received data to the server 140 for analysis, or directly performing analysis. In addition, the user terminal 130 according to an embodiment of the present disclosure can output a training video image to the user 190. Such a training video image may be provided in a form of real-time streaming but a previously recorded and stored video image may be provided. Such a training video image may be output in the form stored in the user terminal 130, but may be output in a form in which the training video image stored in a video image database (to be described below) of the server 140 is provided to the user terminal 130.

According to one embodiment of the present disclosure, the user terminal 130 may correspond to at least one of a mobile phone, a cellular phone, a smart phone, a personal computer, a laptop, a notebook computer, a netbook or tablet, a personal digital assistant (PDA), a digital camera, a game console, an MP3 player, a personal multimedia player (PMP), an electronic book (E-Book), a navigation device, a disk player, a set-top box, a home appliance, a communication device, and a display device.

The server 140 is a configuration unit for exchanging data with the user terminal 130. As an example, the server 140 can receive action data from the user terminal 130 and analyze an action of the user 190.

Home training has a clear advantage of saving time and money, but there is a problem in that it is difficult to induce active participation.

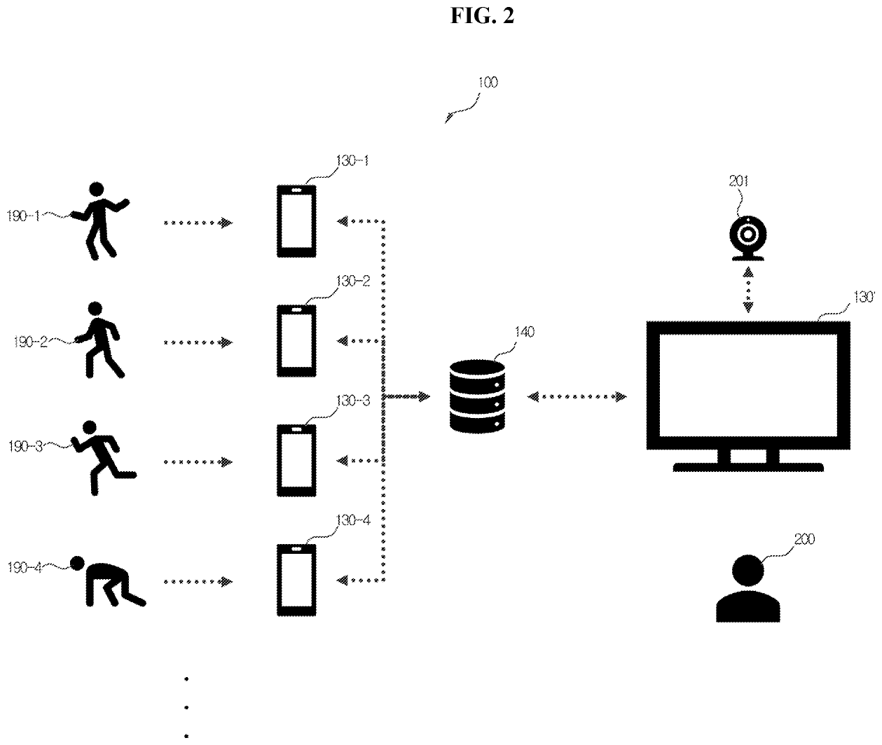

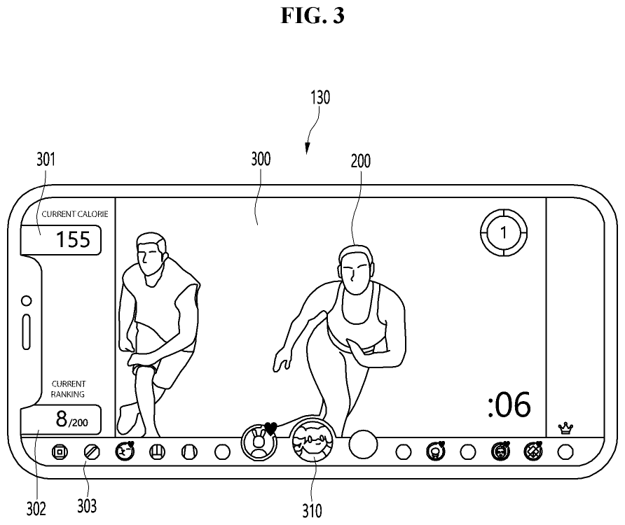

In the present disclosure, in order to induce active participation of the user, a system, in which the user can exercise not only with a trainer but also with other users in a virtual online space even when the user exercises alone at home, is suggested. Through this, the user can participate in the exercise more actively, and the effect of the exercise can be further improved by providing an encouraging feedback to the users who are lagging behind and inducing competition with other users. To this end, the server 140 according to an embodiment of the present disclosure can be provided to exchange data with a plurality of user terminals 130. The concept of interactive will be described in more detail with reference to FIGS. 2 and 3.

FIG. 2 is a conceptual diagram for describing an interactive action in accordance with an exemplary embodiment of the present invention. FIG. 3 illustrates a training screen of the user terminal 130 for implementing the interactive action in accordance with another exemplary embodiment of the present invention. Description will be made with reference to FIGS. 2 and 3 together. In the illustrated drawings, a system including a plurality of user terminals 130-1 to 130-4 is described, and when an individual operation for each of the plurality of user terminals 130-1 to 130-4 is described, the user terminals 130-1 to 130-4 will be referred to as the user terminal 130.

As illustrated in the drawing, the interactive fitness system 100 according to an embodiment of the present disclosure provides a system in which a plurality of users 190-1 to 190-4 can perform home training together. Each of the plurality of users 190-1 to 190-4 can exchange data with the server 140 through each of their user terminals 130-1 to 130-4.

According to an embodiment of the present disclosure, the server 140 can provide a training video image 300 to the plurality of user terminals 130-1 to 130-4. In this case, as described above, the training video image 300 to be provided may be provided in a form of real-time streaming, or a previously recorded and stored image may be provided.

Although the plurality of users 190-1 to 190-4 perform training in their respective homes, each user can expect an effect of participating more actively than when performing the training alone by feeling as if they are training together while viewing the same training video image 300.

In this case, each user 190 can perform training according to the training video image 300 being output while wearing the wearable terminal 120 on his/her smart mat 110. The user terminal 130 may receive action data acquired by the smart mat 110 and the wearable terminal 120 and transmit the action data to the server 140.

When the server 140 receives the action data, the server 140 can check whether or not the user 190 is correctly performing training based on the action data. In addition, the server 140 can provide the user terminal 130 with calorie data obtained by calculating calorie consumption based on the received action data. Based on the calorie data, the user terminal 130 can output calorie consumption information 301.

The server 140 can determine the ranking of each user based on the action data received from the plurality of user terminals 130-1 to 130-4, and provide ranking information therefor to each of the plurality of user terminals 130-1 to 130-4. Each user terminal 130 that has received the ranking information can output current ranking information 302 and all ranking information 303. As in the illustrated example, on all ranking information, the ranking information of other users 190 currently viewing the same training video image 300 is displayed together, and the ranking can be provided in the form of a profile image 310.

Furthermore, the user terminal 130 according to an embodiment of the present disclosure can output a feedback voice based on the received ranking information or point information.

When the training video image 300 is provided in the form of real-time streaming, a trainer 200 can capture the training video image 300 in real time through the camera 201, and provide the captured training image 300 to the server 140.

The server 140 according to an embodiment of the present disclosure, based on the action data received from the plurality of user terminals 130-1 to 130-4, can monitor whether or not the users are performing correctly in real time and can provide a real-time monitoring screen for outputting the monitoring result through a user terminal 130′ of the trainer 200 (hereinafter referred to as a trainer user terminal). The trainer 200 refers to the real-time monitoring screen and delivers praise feedback if there is a user who is performing well in training among the plurality of users 190-1 to 190-4, or points out an incorrect posture or delivers encouraging feedback if there a user who does not follow the training correctly, thereby capable of inducing active participation. Such feedback of the trainer 200 may be transmitted to each user terminal 130 through real-time streaming.

In the illustrated example, the camera 201 is connected to the trainer terminal 130′ to perform photographing for a real-time streaming video image, but is not necessarily limited thereto, and the camera 201 may provide a streaming video image directly to the server 140 without using the trainer terminal 130′.

In the illustrated drawings, although the embodiment in which the training image 300 is provided in the form of real-time streaming has been described, it is obvious that the same can be applied to the embodiment in which the previously recorded image stored in the image DB of the server 140 is provided.

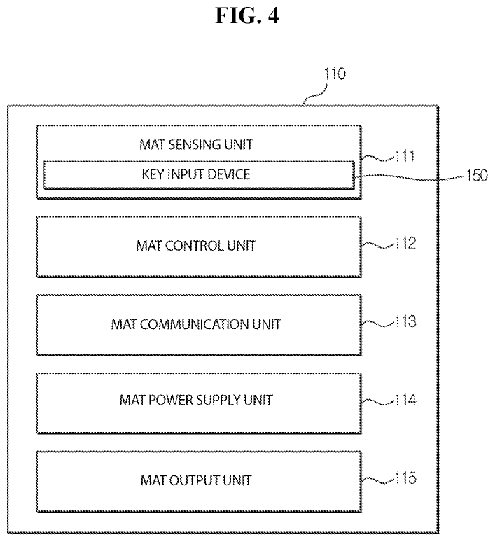

FIG. 4 illustrates a block diagram of the smart mat 110 in accordance with still another exemplary embodiment of the present invention.

The smart mat 110 can include a mat sensing unit 111, a mat control unit 112, a mat communication unit 113, a mat power supply unit 114, a mat output unit 115, etc. The components illustrated in FIG. 4 are not essential in implementing the smart mat 110, and thus the smart mat 110 described in this specification may include more or fewer components than the components listed above.

The mat sensing unit 111 detects contact with at least a part of the body of the user 190. The mat sensing unit 111 detects that the sole, knee, hand, and hip contact the smart mat 110 while the user 190 is training on the smart mat 110. For example, the mat sensing unit 111 may detect this contact through a pressure sensing method. The mat sensing unit 111 according to an embodiment of the present disclosure may detect a contact position and area.

Meanwhile, the mat sensing unit 111 may be configured to include the key input device 150 according to an embodiment of the present disclosure, which will be described later. The key input device 150 is a device that divides the entire region of the smart mat 110 into a plurality of sub-regions, and determines whether or not contact occurs in the divided sub-regions.

The mat control unit 112 controls the overall operation of the smart mat 110. The mat control unit 112 may judge whether or not the user 190 is performing training correctly based on the action data. It is only an example that such judgment is made on the smart mat 110, and the judgment may be made by at least one of the smart mat 110, the user terminal 130, and the server 140.

The mat communication unit 113 is a configuration unit for exchanging data with at least one of the wearable terminal 120, the user terminal 130, and the server 140.

The mat power supply unit 114 supplies power to each component included in the smart mat 110 by receiving external power and internal power under the control of the mat control unit 112. The mat power supply 114 includes a battery, and the battery may be a built-in battery or a replaceable battery.

The mat output unit 115 can output the judgment result as to whether or not the user 190 is performing training correctly. As described above, the judgment output through the mat output unit 115 may be judgment made by the mat control unit 112, but may also be judgment made by the server 140 or other terminals 120 and 130. For example, a feedback voice can be output through the mat output unit 115.

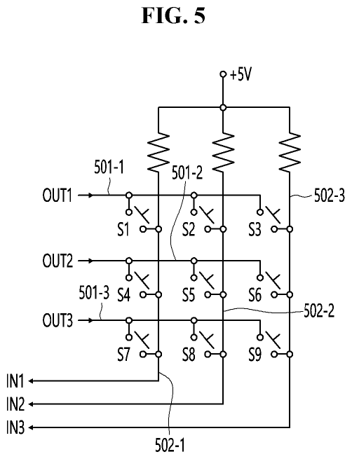

Prior to describing a specific embodiment of the key input device 150, a key detection method through a key matrix structure will be described with reference to FIG. 5.

FIG. 5 is a diagram illustrating a general key detection method based on a key matrix structure. FIG. 5 is an exemplary diagram of a simple key matrix circuit, and for a case of a simple key matrix having 9 keys, a basic principle of the key matrix detection method is described based on the example.

As illustrated, when three horizontal row electrode lines 501-1 to 501-3 and three vertical column electrode lines 502-1 to 502-3 meet, positions of nine switches S1 to S9 are determined at the points where the electrode lines cross each other. The row electrode lines 501-1 to 501-3 are respectively connected to output terminals of OUT 1 to OUT 3, and the column electrode lines 502-1 to 502-3 are respectively connected to input terminals of IN 1 to IN 3. When a predetermined key is pressed, a switch corresponding to the key operates to close. Hereinafter, pressing a key and switching (or switching operation) are used as having the same meaning.

A processor embedded in the key input device scans the input terminals of IN 1 to IN 3 while sequentially applying a low potential signal (logic 0) to the output terminals of OUT 1 to OUT 3. A high potential signal (logic 1) is output from each input terminal when the key is not pressed and a low potential signal is output when the key is pressed, and thus it may be detected that a key at a point where the output terminal to which the low potential signal is applied and the input terminal to which the low potential signal is input cross is pressed. The processor can check which key is pressed through such a key scan process.

To explain this in more detail, the processor sends logic 0 to OUT 1 and reads IN 1 to 3, then sends logic 0 to OUT 2 again and reads IN 1 to 3 again. After that, the processor sends logic 0 to OUT 3 again, reads IN 1 to 3, and then repeats the process of sending logic 0 to OUT 1 to 3 again to scan whether or not the key is pressed.

In this process, if no key is pressed, logic 1 is measured at IN 1 to 3. However, when any key is pressed, there are places where logic 0 is measured at IN 1 to 3 to which logic 0 is input. If logic 0 is measured at IN 1, it indicates that one of S1, S4, and S7 is pressed. When IN 2 becomes logic 0, it indicates that one of S2, S5, and S8 is pressed. When IN 3 becomes 0, it indicates that one of S3, S6, and S9 is pressed.

In order to know which key is pressed, it is needed to check which of OUT 1 to 3 is applied with logic 0. Assuming that IN 2 becomes logical 0 when sending logic 0 to OUT 2, S5 is the only point where OUT 2 and IN 2 meet. In the end, it can be seen that S5 has been pressed.

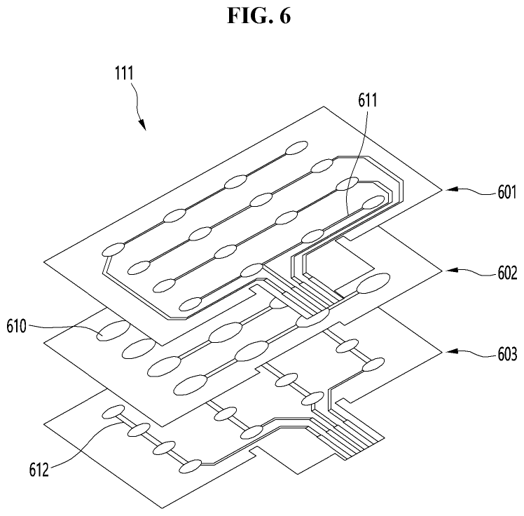

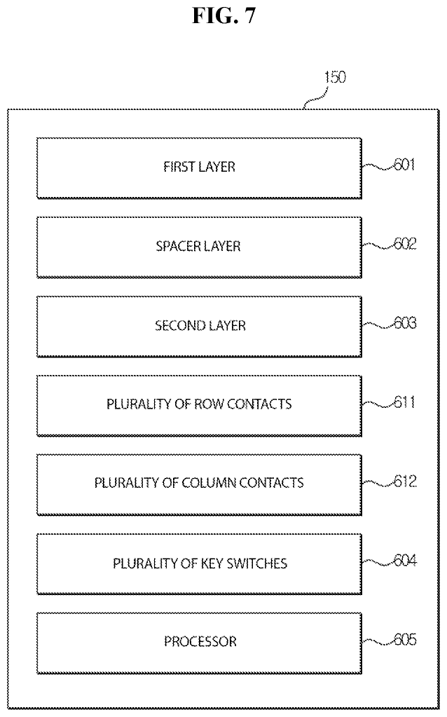

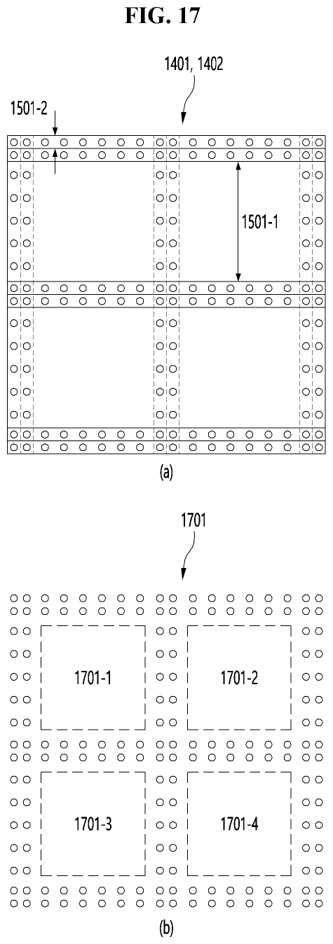

FIG. 6 illustrates a stacked structure of a key input device 150 in accordance with still another exemplary embodiment of the present invention. FIG. 7 illustrates a block diagram of the key input device 150 in accordance with still another exemplary embodiment of the present invention. In a first embodiment related to FIG. 7, a spacer layer 602 is inserted between a first and second layers 601 and 603, but this is one embodiment, and the present disclosure is limited to this form. In a second embodiment, which will be described later with reference to FIG. 17, a form of spacer dots may be provided instead of the spacer layer 602.

The key input device 150 according to an embodiment of the present disclosure can be configured to include the first layer 601, the spacer layer 602, the second layer 603, a plurality of row contacts 611, and a plurality of column contacts 612, a plurality of key switches 604, and a processor 605. The components illustrated in FIG. 7 are not essential for implementing the key input device 150, and thus the key input device 150 described herein can include more or fewer components than those listed above.

The key input device 150 according to an embodiment of the present disclosure can be configured to include the first layer 601 forming the plurality of row contacts 611 and the second layer 603 forming the plurality of column contacts 612. The first and second layers 601 and 603 may be provided to overlap each other so that the plurality of column and row contacts 611 and 612 formed in respective layers cross each other.

The plurality of row and column contacts 611 and 612 may perform operations corresponding to the row and column electrode lines 501 and 502 in FIG. 5 described above. That is, each of the plurality of row contacts 611 can be connected to the processor 605 through an output terminal, and each of the plurality of column contacts 612 can be connected to the processor 605 through an input terminal. The processor 605 may check which key (or key region) is pressed through the key scan process described above with reference to FIG. 5.

For example, in the first and second layers 601 and 603, the plurality of row and column contacts 611 and 612 can be formed by printing with conductive ink on a thin film made of a material such as PE. The spacer layer 602 may be further provided between the first and second layers 601 and 603 so as to prevent contact between the plurality of row and column contacts 611 and 612 in a state where there is no contact or no pressure is applied.

Each of the plurality of row and column contacts 611 and 612 according to an embodiment of the present disclosure can include a plurality of row electrode lines and a plurality of column electrode lines.

The spacer layer 602 according to an embodiment of the present disclosure can form a hole 610 in each of a plurality of key regions, and may block contact between the first and second layers 601 and 603 except for the plurality of key regions. Also, when contact is made or pressure is applied by the user, the row and column contacts 611 and 612 may contact each other through the hole formed in the key region.

The key region according to an embodiment of the present disclosure is a point where each of the plurality of row contacts 611 and each of the plurality of column contacts 612 cross, and may refer to a region where the row and column contacts 611 and 612 come into contact and a switching operation occurs.

A plurality of key switches 604 are formed by the plurality of row contacts 611 and the plurality of column contacts 612. More specifically, the plurality of key switches 604 can be provided in a plurality of key regions in such a way that the row and column contacts 611 and 612 contact each other or separate from each other according to the user's contact or pressurization.

The processor 605 detects that at least one of the plurality of key switches 604 is pressed. Here, the fact that the key switch 604 is pressed may be a switching operation. That is, the processor 605 may detect the switching operation made on at least one of the plurality of key switches 604.

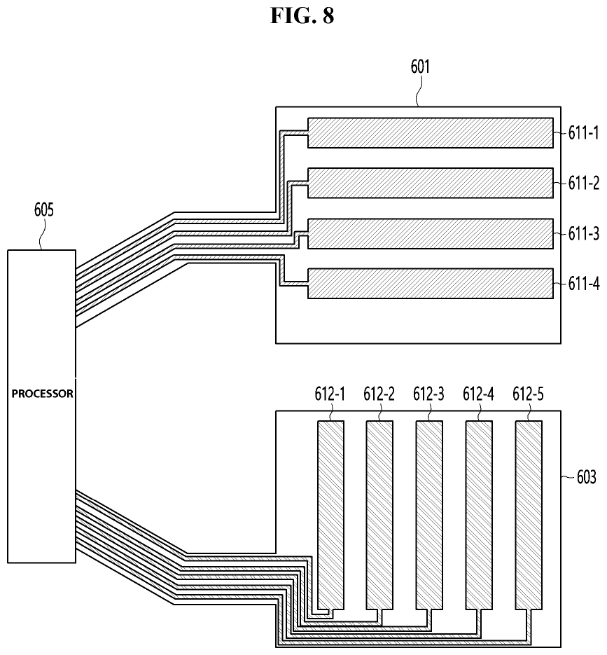

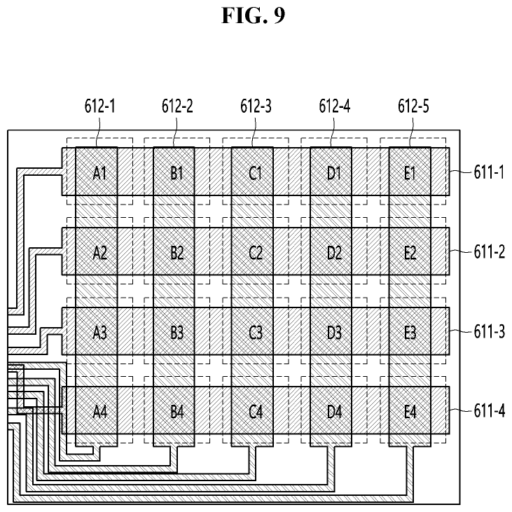

Hereinafter, a plurality of key regions formed based on the row and column contacts 611 and 612 according to an embodiment of the present disclosure will be described with reference to FIGS. 8 and 9.

FIG. 8 illustrates a conceptual diagram in which the plurality of row and column contacts 611 and 612 are formed in accordance with still another exemplary embodiment of the present invention. FIG. 9 illustrates key regions A1 to E4 formed by the plurality of row and column contacts 611 and 612 in accordance with still another exemplary embodiment of the present invention. Description will be made with reference to FIGS. 8 and 9 together.

In the illustrated drawing, the plurality of row contacts 611 are illustrated as four row contacts and the plurality of column contacts 612 are illustrated as five column contacts, but the present disclosure may not be limited to these numbers of contacts.

Although the first layer 601 and the second layer 603 are illustrated separately from each other in FIG. 8, it is obvious that the first and second layers 601 and 603 may be provided in an overlapped state as in FIG. 9.

Each of the plurality of row contacts 611-1 to 611-4 is connected to the processor 605 through the output terminal. Similarly, each of the plurality of column contacts 612-1 to 612-5 is connected to the processor 605 through the input terminal.

As illustrated in FIG. 9, a plurality of key regions A1 to E4 may be formed at points where the plurality of row and column contacts 611 and 612 cross each other. When assuming that the number of the plurality of row contacts 611 is N and the number of the plurality of column contacts 612 is M, the number of the plurality of key regions A1 to E4 to be formed may be as many as N×M.

For example, when a user's contact occurs in the key region A1, the processor 605 may detect (as in the key scan process in FIG. 5) that a current is carried between the first row contact 611-1 and the first column contact 612-1. The processor 605 may determine that contact has occurred in the key region A1, which is a point where the first row contact 611-1 and the first column contact 612-1 cross each other, based on the detection result as described above.

When the key scanning process described above is repeatedly performed on the plurality of key regions A1 to E4, a detecting operation for the body of the user 190 may be performed on the smart mat 110 described above in FIG. 4.

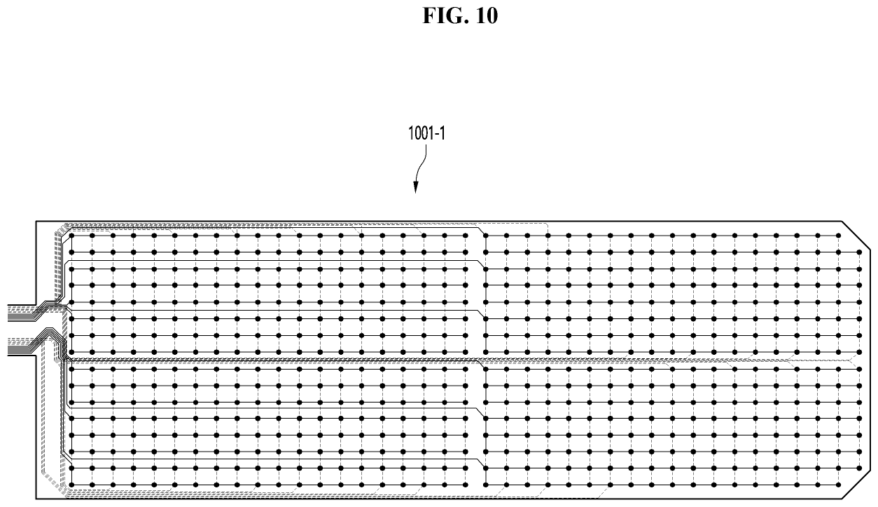

Meanwhile, in order to effectively detect the contact made on the key region, a shape or form of the plurality of row and column contacts 611 and 612 may be important. The shape or form of the row and column contacts 611 and 612 thus formed is hereinafter referred to as a contact pattern. Hereinafter, the contact pattern will be described in more detail with reference to FIGS. 10 to 13.

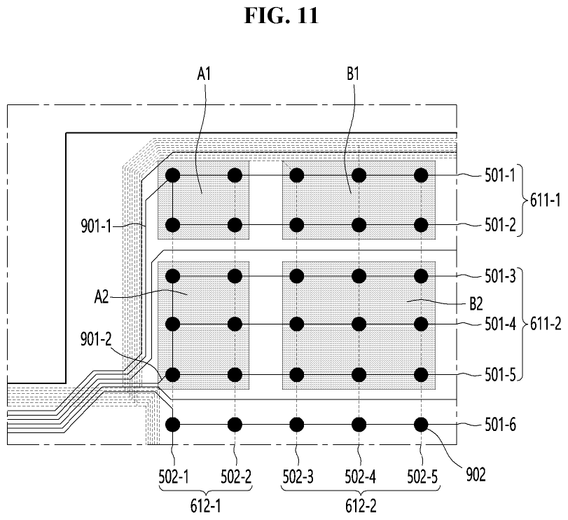

FIG. 10 is a diagram illustrating a first contact pattern 1001-1 in accordance a first embodiment of the present invention. FIG. 11 is an enlarged diagram of a part of the first contact pattern 1001-1 of FIG. 10. Description will be made with reference to FIGS. 10 and 11 together.

In FIGS. 10 and 11, the plurality of row contacts 611 are illustrated by solid lines, and the plurality of column contacts 612 are illustrated by dotted lines. The row and column contacts 611 and 612 are formed on different layers and are kept spaced apart by a predetermined distance by the spacer layer 602 described above, and thus the row and column contacts 611 and 612 may appear superimposed on the drawing but remain uncontacted in practice.

A plurality of key regions A1 to B2 may be formed on the first contact pattern 1001-1.

The first contact pattern 1001-1 according to a first embodiment of the present invention is suggested to be formed with a plurality of row or column electrode lines 501-1 to 501-5. As illustrated in FIG. 11, the first row contacts 611-1 are formed with first and second row electrode lines 501-1 and 501-2 arranged in parallel. In addition, the second row contacts 611-2 are formed with third to fifth row electrode lines 501-3 to 501-5 arranged in parallel.

Similarly, the first column contact 611-1 is formed with first and second column electrode lines 502-1 and 502-2 arranged in parallel. In addition, the second row contact 611-2 is formed with third to fifth row electrode lines 502-3 to 502-5 arranged in parallel.

Referring to the illustrated drawing, a key region is formed in a region where one row contact and one column contact overlap. For example, the key region A1 is formed in a region where the first row contact 611-1 and the first column contact 612-1 overlap.

The number of points where the contacts cross each other (hereinafter referred to as intersecting points) in the key region may vary according to the number of electrode lines forming each contact. For example, in the key region A1, two row electrode lines 501-1 and 501-2 and two column electrode lines 502-1 and 502-2 meet to form four intersecting points. In the key region B2, three row electrode lines 501-3 to 501-5 and three column electrode lines 502-3 to 502-5 meet to form nine intersecting points.

A plurality of intersecting points are formed on one key region, but the same key signal may be generated on the same key region regardless of which of the plurality of intersecting points the switching operation (contact between row and column electrode lines) occurs.

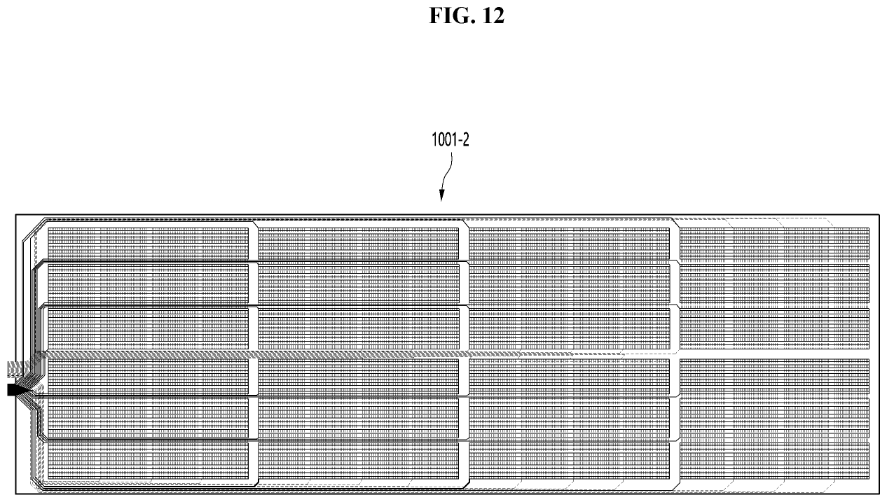

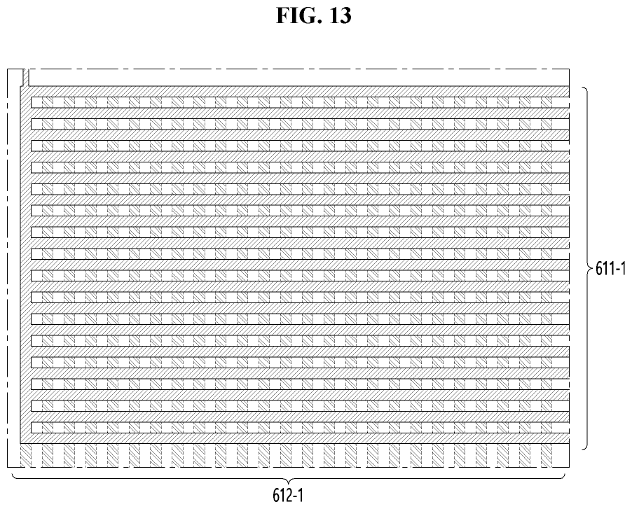

FIG. 12 is a diagram illustrating the second contact pattern 1001-2 in accordance with a second embodiment of the present invention. FIG. 13 is an enlarged diagram of a part of the second contact pattern 1001-2 of FIG. 12. Description will be made with reference to FIGS. 12 and 13 together.

In FIG. 13, the plurality of row contacts 611 are illustrated in a diagonal pattern, and the plurality of column contacts 612 are illustrated in a double diagonal pattern. The row and column contacts 611 and 612 are formed on different layers and are kept spaced apart by a predetermined distance by the spacer layer 602 described above, and thus the row and column contacts 611 and 612 may appear superimposed on the drawing but remain uncontacted in practice.

The second contact pattern 1001-2 according to a second embodiment of the present disclosure is suggested to be formed with a plurality of row or column electrode lines.

Unlike in the first embodiment described above, the second contact pattern 1001-2 according to the second embodiment may be formed more densely (with a narrow interval between adjacent electrode lines) with a relatively larger number of row or column electrode lines than in the first embodiment.

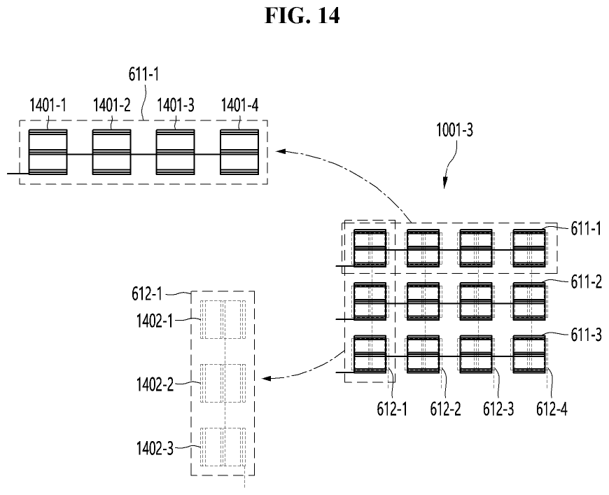

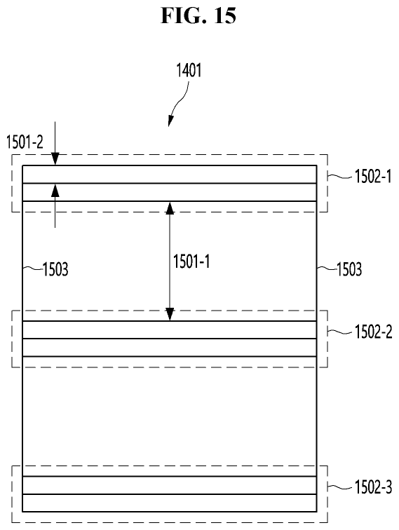

Meanwhile, according to the contact pattern 1101-2 of FIGS. 12 and 13, there is a problem in that, it is erroneously recognized that adjacent key switches are pressed together even though one key switch is pressed. This misrecognition is analyzed to be due to parasitic capacitance generated by the relatively densely provided electrode lines. Hereinafter, a contact pattern 1101-3 in FIGS. 14 and 15 are suggested in order to solve such a problem.

FIG. 14 is a diagram illustrating a third contact pattern 1001-3 in accordance with a third embodiment of the present invention. FIG. 15 is a diagram illustrating a shape of a row contact unit 1401 in accordance with still another exemplary embodiment of the present invention

As in the illustrated drawing, the plurality of row contacts 611 may be configured to include a plurality of row contact units 1401-1 to 1401-4. In addition, the plurality of row contact units 1401-1 to 1401-4 included in one row contact 611 may be connected to the processor 605 through one output terminal by being connected to each other.

As illustrated, the plurality of row contact units 1401-1 to 1401-4 included in the first row contact 611-1 are connected to each other through an electrode line. The plurality of row contact units 1401-1 to 1401-4 connected in this way may be connected to the processor 605 through one output terminal.

Similarly, the plurality of column contacts 612 may be configured to include a plurality of column contact units 1402-1 to 1402-4. In addition, the plurality of column contact units 1402-1 to 1402-4 included in one column contact 612 may be connected to the processor 605 through one input terminal by being connected to each other.

As illustrated, the plurality of column contact units 1402-1 to 1402-4 included in the first column contact 612-1 are connected to each other through an electrode line. The plurality of column contact units 1402-1 to 1402-4 connected in this way may be connected to the processor 605 through one input terminal.

Furthermore, the number of row contact units 1401-1 to 1401-4 according to an embodiment of the present invention is suggested to coincide with the number of the plurality of column contacts 612. In the illustrated FIG. 14, since the number of column contacts 612 is four, the number of row contact units 1401-1 to 1401-4 to be provided may also be four.

Similarly, the number of column contact units 1402-1 to 1402-3 according to an embodiment of the present invention is suggested to coincide with the number of row contacts 611. In the illustrated FIG. 14, since the number of row contacts 611 is three, the number of column contact units 1402-1 to 1402-3 to be provided may also be three.

The shape of the row contact unit 1401 will be described in detail with reference to FIG. 15. In FIG. 15, description will be made with the row contact unit 1401 as a reference, but when rotating the row contact unit 1401, the row contact unit may be in the shape of the column contact unit 1402.

The row contact unit 1401 according to an embodiment of the present invention includes a plurality of electrode line bundles 1502-1 to 1502-3 arranged at a first interval 1501-1 and a connection line 1503 connecting the plurality of electrode line bundles 1502-1 to 1502-3, and the electrode line bundle includes a plurality of electrode lines arranged at a second interval 1501-2.

Referring to the shape of the illustrated row contact unit 1401, a pattern having an ‘empty space (space between electrode line bundles having the first interval)’ therein reduces an area of the entire conducting line, and thus misrecognition due to parasitic capacitance can be minimized.

Meanwhile, the first to third contact patterns 1001-1 to 1001-3 described above may be suitable to cover a certain area, and may be somewhat insufficient to cover the entire area of the smart mat 110 according to an embodiment of the present invention. To this end, in one embodiment of the present invention, it is suggested to divide the entire area of the smart mat 110 into a plurality of regions, and to apply the first to third contact patterns described above to each divided region.

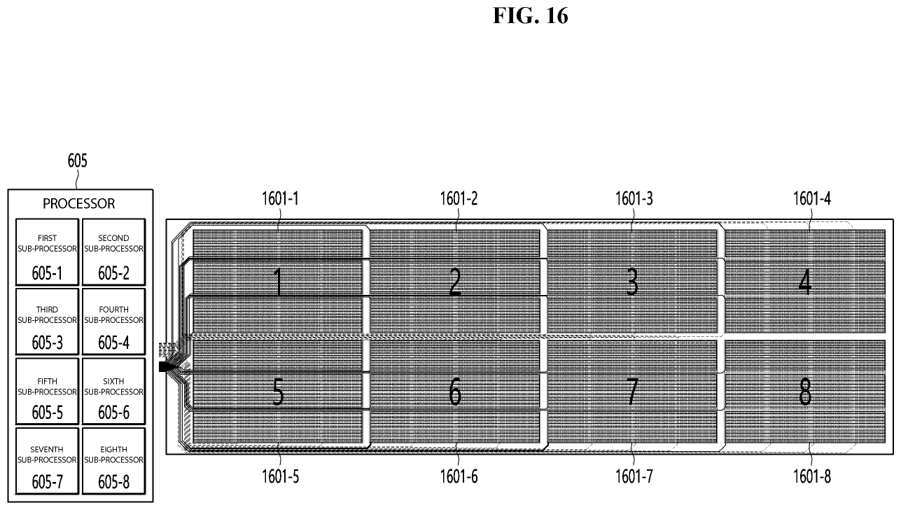

FIG. 16 illustrates a conceptual diagram for dividing the entire area of the smart mat 110 in accordance with still another exemplary embodiment of the present invention. According to the illustrated drawing, the entire area of the smart mat 110 is divided into eight regions 1601-1 to 1601-8, but the present invention is not limited to this number of eight regions.

The processor 605 is configured to include a plurality of sub-processors 605-1 to 605-8 for respectively processing the plurality of regions 1601-1 to 1601-8. The plurality of sub-processors 605-1 to 605-8 are connected to the plurality of regions 1601-1 to 1601-8, respectively, and individually detect a contact made on the divided area. In addition, the plurality of sub-processors 605-1 to 605-8 transmits the detection result to the processor 605.

The processor 605 detects contact of a user's body made on the entire smart mat 110 based on the detected results from the plurality of sub-processors 605-1 to 605-8.

When each of the plurality of regions 1601-1 to 1601-8 is processed in this way, there is an effect that the sensing speed for each region can be increased, and sensing resolution for each individual region is improved. This is because the number of key regions that can be processed by one processor (the number of key regions that can be simultaneously processed) is limited.

Meanwhile, the smart mat 110 according to an embodiment of the present invention can be stored in the form of rolling the mat during storage (when not exercising). Accordingly, in an embodiment of the present invention, the smart mat 110 is suggested to have a layered structure that is easy to store.

On the other hand, as in the embodiment described above with FIG. 7, when the smart mat is provided in a form in which several layers 601, 602, and 603 overlap, the smart mat may not be easy to roll. This is because, when rolled, a gap occurs between the inner layer and the outer layer due to the difference in radius between the layers.

In order to solve this problem, in the second embodiment of the present invention, instead of providing the spacer layer 602, it is suggested to form spacer dots on any one of the first and second layers 601 and 603. That is, the spacer dots serving as the spacer layer 602 are provided in one of the first and second layers 601 and 603.

FIG. 17 illustrates a spacer dot pattern 1701 formed in the row/column contact units 1401 and 1402 in accordance with still another exemplary embodiment of the present invention. In order to prevent the row/column contact units 1401 and 1402 from contacting each other, the spacer dot pattern 1701 is formed such that the spacer dots are formed between the plurality of electrode line bundles 1502-1 to 1502-3.

More specifically, the spacer dot pattern 1701 may be formed such that the spacer dots are provided between the plurality of electrode line bundles 1502-1 to 1502-3 formed at the second interval 1501-2.

When the spacer dots are formed in this way, spacer dot blanks 1701-1 to 1701-4 may be formed between the plurality of electrode line bundles formed at the first interval 1501-1 in the center.

Meanwhile, it is suggested that the first and second layers 601 and 603 are adhered to each other. This adhesion method will be described in more detail with reference to the drawings below.

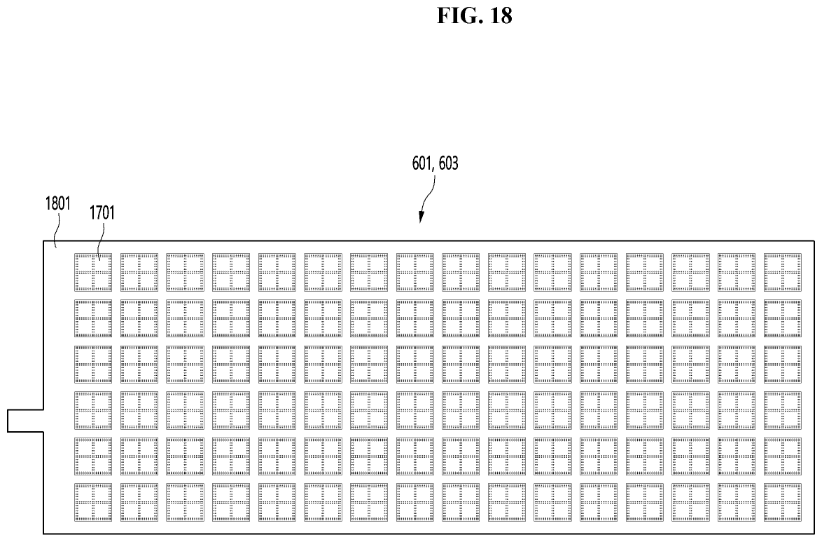

FIG. 18 illustrates an insulating structure of the first and second layers 601 and 603 in accordance with still another exemplary embodiment of the present invention.

According to an embodiment of the present disclosure, when adhering the first and second layers 601 and 603, it is suggested to adhere the first and second layers 601 and 603 to each other after insulating the remaining portions except for the key region. That is, an insulating layer 1801 is formed between the first and second layers 601 and 603, and the insulating layer 1801 is insulated from the remaining regions except for the key region 1701 described above.

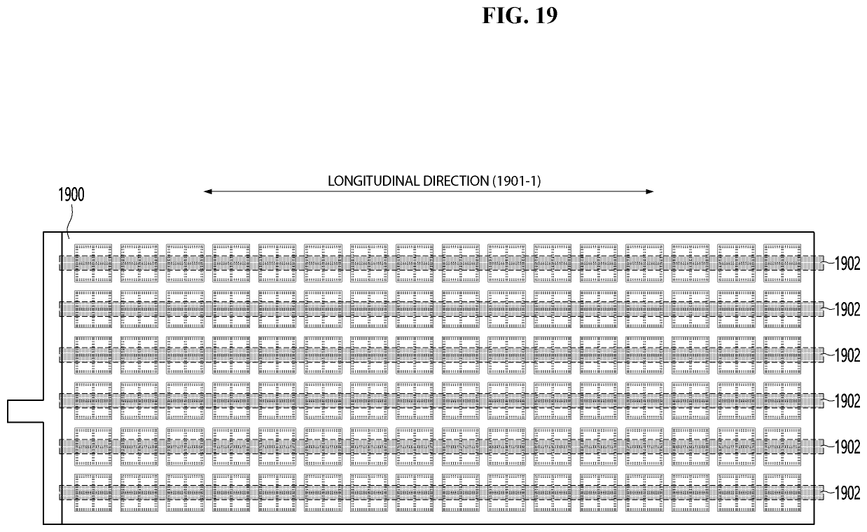

FIG. 19 illustrates the adhesive structure of the first and second layers 601 and 603 in accordance with still another exemplary embodiment of the present invention. When the first and second layers 601 and 603 described above are adhered to each other, adhesive may need to be applied to the first and second layers 601 and 603. A layer formed by the adhesive applied in this way is referred to as an adhesive layer 1900.

The first and second layers 601 and 603 according to an embodiment of the present disclosure are suggested to be adhered to each other by forming air holes. This is because, when the smart mat 110 according to an embodiment of the present disclosure is rolled up for storage, it is to minimize the phenomenon that the mat is wrinkled by air remaining between the layers.

In particular, as illustrated in FIG. 19, the first and second layers 601 and 603 are suggested to form an air path 1902 in a longitudinal direction 1900-1 of the smart mat 110. In an embodiment of the present invention, the adhesive layer 1900 may be formed in a state in which air holes are respectively formed at the positions of the plurality of row contacts in the longitudinal direction 1901-1. In this case, the longitudinal direction 1901-1 may mean a relatively longer direction among the horizontal and vertical directions of the smart mat 110.

Furthermore, a plurality of air paths 1902 according to an embodiment of the present disclosure may be provided as many as possible to pass through all of the plurality of key regions provided in the smart mat 110. According to the example illustrated in FIG. 19, since the key region configuring the smart mat 110 is configured with six rows, six air paths 1902 can be formed. In addition, the six air paths 1902 may be formed to pass through all of the key regions all of which are composed of six rows in the longitudinal direction 1901-1.

Also, the adhesive layer 1900 may be adhered in the remaining region except for the key region described above. When the adhesive treatment is made on a fairly wide region of the smart mat 110 in this way, the sound of bumping between films (layers) and the phenomenon that films (layers) are wrinkled may be minimized.

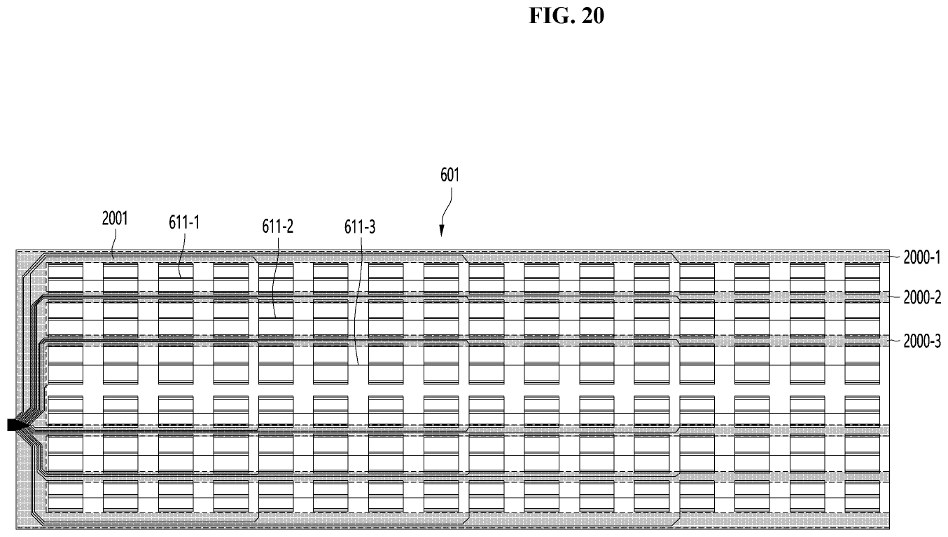

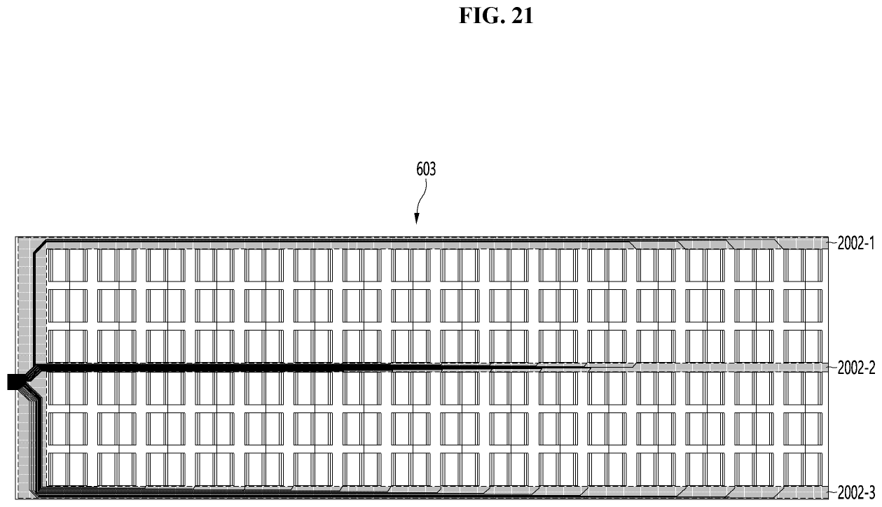

Meanwhile, for the smart mat 110 according to an embodiment of the present disclosure, a structure capable of minimizing a dead space at which a contact with the user's body is not recognized is suggested. This structure will be described with reference to FIGS. 20 and 21 together.

FIG. 20 is a diagram illustrating a row dead space 2001 for a plurality of row contacts formed on the first layer 601 in accordance with still another exemplary embodiment of the present invention. FIG. 21 is a diagram illustrating a column dead space 2002 for a plurality of column contacts formed on the second layer 603 in accordance with still another exemplary embodiment of the present invention.

As described above, the plurality of row and column contacts 611 and 612 should be connected to the processor 605 through input and output terminals. An electrode line for connection may be formed differently depending on the position of the processor 605.

FIGS. 20 and 21 are diagrams when the processor 605 is on the left side of the drawing. Electrode lines may be formed in all of the plurality of row and column contacts 611 and 612 formed in the first and second layers 601 and 603 in order for the plurality of row and column contacts 611 and 612 to be connected to the processor 605 positioned on the left side.

In this case, not only the left region adjacent to the processor 605 but also the right region adjacent thereto should form an electrode line with a left end.

As described above with reference to FIG. 16, when the entire region of the smart mat 110 is divided into a plurality of sub-regions, an electrode line for each of the divided regions may occupy a significant portion of the area of the smart mat 110.

When the electrode line is formed long in the left or right direction as described above, the region through which the electrode line passes will become a dead space in which a user's touch is not detected.

If the area of the dead space is large, the possibility of causing misrecognition may increase.

Accordingly, in an embodiment of the present invention, a structure in which electrode lines can be appropriately distributed is suggested.

Among the plurality of regions divided in FIG. 16, a first to fourth regions 1601-1 to 1601-4 arranged above will be described. The arrangement of the electrode lines in the remaining fifth to eighth regions 1601-5 to 1601-8 may be provided to be symmetrical to the arrangement of the electrode lines in the first to fourth regions 1601-1 to 1601-4.

A first electrode line 2000-1 for the first row contact 611-1 positioned at the top of the plurality of row contacts 611 provided in each of the first to fourth regions 1601-1 to 1601-4 is formed to pass above the first row contact 611-1.

A second electrode line 2000-2 for the second row contact 611-2 positioned immediately below the first row contact 611-1 can be formed to pass between the first and second row contacts 611-1 and 611-2. Similarly, a third electrode line 2000-3 for the third row contact 611-3 positioned immediately below the second row contact 611-2 may be formed to pass between the second and third row contacts 611-2 and 611-3.

In the illustrated example, although a structure having the first to third row contacts 611-1 to 611-3 is described, but even if the number row contacts is increased, the electrode lines may be expanded and formed in the same manner.

Referring to FIG. 21, when the area of the smart mat is divided into an upper region (first to fourth regions 1601-1 to 1601-4) and a lower region (fifth to eighth regions 1601-5 to 1601-8), the electrode line may be formed in an upper portion 2002-1 of the upper region, a middle portion 2002-2 between the upper region and the lower region, and a lower portion 2002-3 of the lower region.

Meanwhile, in the case of the key input device according to an embodiment of the present invention, in order to be applied to a shape such as a smart mat, there is a task to be able (1) to detect the user's light touch and (2) to minimize the probability of being misrecognized even if the mat is rolled and unfolded repeatedly.

In addition, there is a problem that when the mat stored in a dried state is unfolded, the part that was in contact between the row contact 611 and the column contact 612 has to come off while being restored by elasticity of the mat, but due to lack of the elasticity of the mat, the contact state continues and the misrecognition occurs.

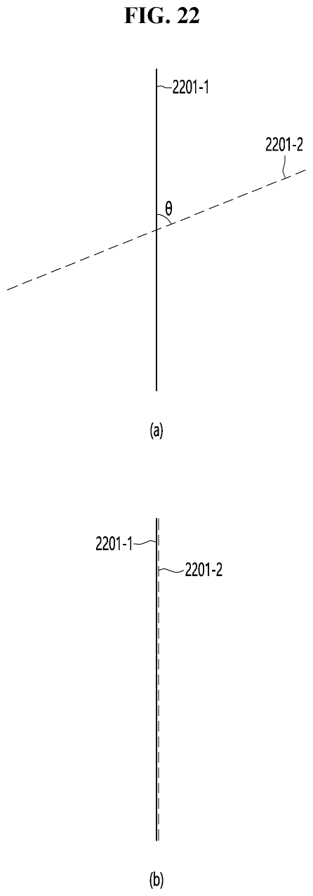

FIG. 22 is a conceptual diagram for describing a shape in which electrode lines cross each other.

The probability of the misrecognition may vary depending on the shape in which electrode lines 2201-1 and 2201-2 that cross each other and constitute the row contact unit 1401 and the column contact unit 1402 according to the embodiment described above.

The shape in which electrode lines cross each other may include a case where the two electrode lines 2201-1 and 2201-2 form a predetermined angle θ and cross each other at one point (hereinafter referred to as point-crossing) and a case where the two electrode lines 2201-1 and 2201-2 may be arranged in the same direction and cross each other along a line (hereinafter referred to as line-crossing).

When considering a contact area, the line-crossing is inevitably wider than the point-crossing, and thus in the case of the line-crossing, the possibility of misrecognition will increase as the contact area increases.

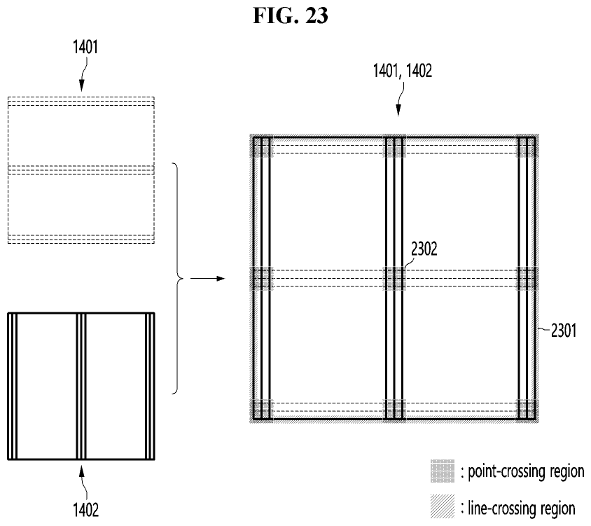

FIG. 23 is a diagram illustrating an example of a shape in the row/column contact units 1401 and 1402 cross each other in accordance with still another exemplary embodiment of the present invention. The form of the row/column contact units 1401 and 1402 is as described above in conjunction with FIG. 15.

In FIG. 23, the horizontal length of the row contact unit 1401 and the horizontal length of the column contact unit 1402 are the same, and the vertical length of the row contact unit 1401 and the vertical length of the column contact unit 1402 are the same. Alternatively, the row/column contact units 1401 and 1402 may be squares of the same size.

In this case, a line-crossing region 2301 may have a rectangular shape forming outer shells of the row/column contact units 1401 and 1402. In addition, a total of nine point-crossing regions 2302 may be arranged in a 3×3 checkerboard shape.

The line-crossing region 2301 refers to a region in which the line-crossing mainly occurs as described above, and the wider the region, the higher the possibility of occurrence of the misrecognition problem described above.

Therefore, in the present invention, which will be described later, a structure of the row/column contact units 1401 and 1402 to avoid line-crossing and induce point-crossing is proposed.

Examples described below may be applied individually, respectively, but the case where at least one or more examples are used in combination will also be included in the present invention.

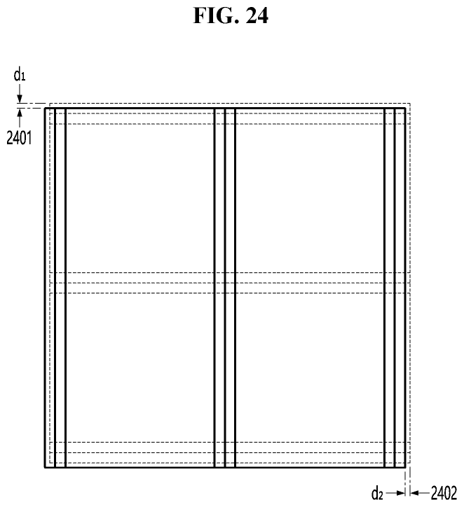

FIG. 24 illustrates a first example of a crossing structure of the row/column contact units 1401 and 1402 in accordance with still another exemplary embodiment of the present invention.

In the first example, the horizontal length of the row contact unit 1401 and the horizontal length of the column contact unit 1402 are the same, and the vertical length of the row contact unit 1401 and the vertical length of the column contact unit 1402 are the same, but is not necessarily limited thereto. The row contact unit 1401 and the column contact unit 1402 having other sizes may be provided.

In the crossing structure of the first example, it is proposed that the array of the row contact unit 1401 and the column contact unit 1402 is arranged to be displaced by a first interval (d₁, 2401) in the horizontal direction, and arranged to be displaced by a second interval (d₂, 2402) in the vertical direction to prevent line-crossing from occurring.

That is, the array of the row contact unit 1401 and the column contact unit 1402 according to an embodiment of the present invention may be arranged to be displaced by the first interval (d₁, 2401) in the horizontal direction, and arranged to be displaced by the second interval (d₂, 2402).

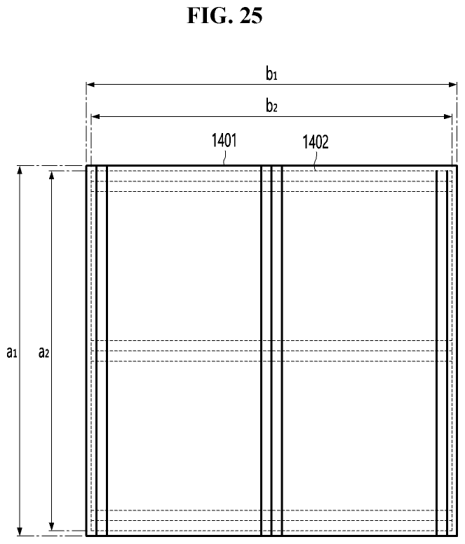

FIG. 25 illustrates a second example of the crossing structure of the row/column contact units 1401 and 1402 in accordance with still another exemplary embodiment of the present invention.

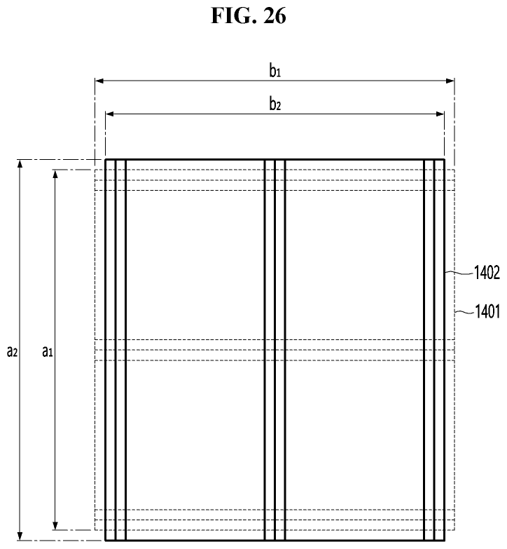

FIG. 26 illustrates a third example of the crossing structure of the row/column contact units 1401 and 1402 in accordance with still another exemplary embodiment of the present invention.

In the second and third examples, a horizontal length b₁ of the row contact unit 1401 and a horizontal length b₂ of the column contact unit 1402 are different from each other, and a vertical length a₁ of the row contact unit 1401 and a vertical length a₂ of the column contact unit 1402 may be different from each other.

In the second example illustrated in FIGS. 25, a₁>a₂ and b₁>b₂, but the present invention is not limited thereto, and a case of different lengths such as a₁<a₂ or b₁<b₂ may also be included in the present invention. Third example illustrated in FIG. 26 is a case where a₁<a₂ and b₁>b₂.

That is, the row contact unit 1401 and the column contact unit 1402 according to an embodiment of the present invention may be provided such that the horizontal length b₁ of the row contact unit 1401 and the horizontal length b₂ of the column contact unit 1402 are different from each other, and the vertical length a₁ of the row contact unit 1401 and the vertical length a₂ of the column contact unit 1402 are different from each other.

On the other hand, instead of a crossing structure using the size or arrangement of the row/column contact units 1401 and 1402, a crossing structure in which the line-crossing region 2301 is eliminated or minimized using the insulating layer 1801 described above is proposed.

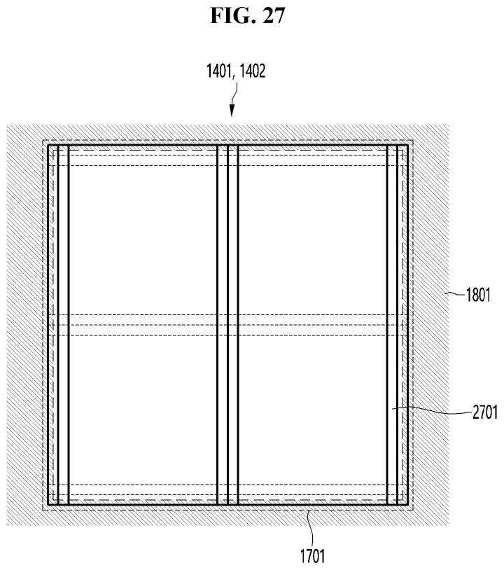

FIG. 27 illustrates a fourth example of the crossing structure of the row/column contact units 1401 and 1402 in accordance with still another exemplary embodiment of the present invention.

In the fourth example, the horizontal length of the row contact unit 1401 and the horizontal length of the column contact unit 1402 may be the same, and the vertical length of the row contact unit 1401 and the vertical length of the column contact unit 1402 may be the same.

In the present invention, a region insulated by the insulating layer 1801 is referred to as an insulating region, and a region that is not insulated because a hole is formed in the insulating layer 1801 is referred to as a current carrying region.

As illustrated in FIG. 23, the line-crossing region 2301 has a rectangular shape forming the outer shells of the row/column contact units 1401 and 1402, and a current carrying region 2701 corresponding to a key region 1701 of the insulating layer 1801 may also have a rectangular shape. That is, the insulating layer 1801 may have a form that partially invades the key region 1701 described above with reference to FIG. 18.

The current carrying region 2701 according to an embodiment of the present invention is formed to correspond to the at least one key region 1701.

The insulating region of the insulating layer 1801 according to an embodiment of the present invention is formed so that the row contact unit and the column contact unit insulate the line-crossing region 2301.

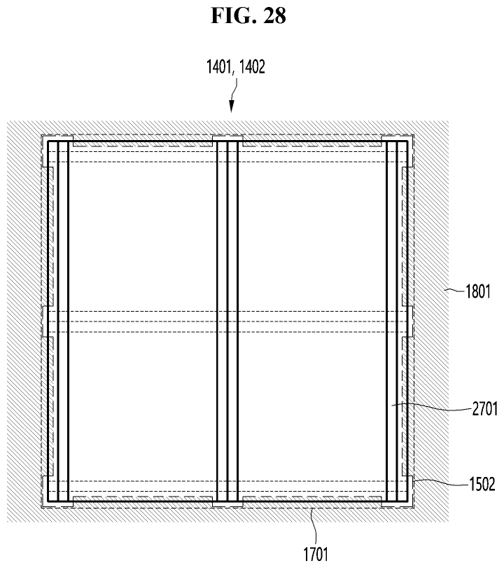

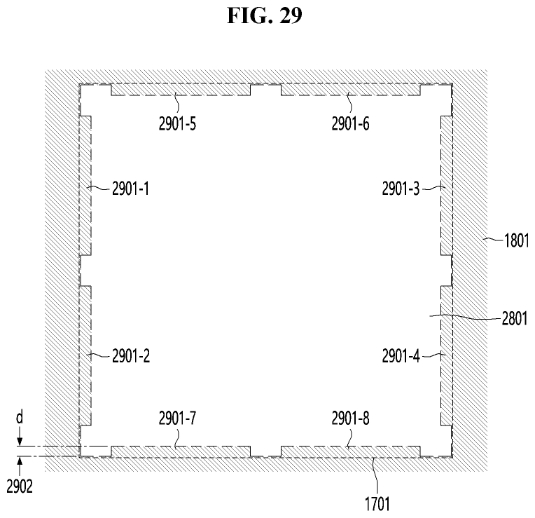

FIG. 28 illustrates a fifth example of the crossing structure of the row/column contact units 1401 and 1402 in accordance with still another exemplary embodiment of the present invention. FIG. 29 illustrates the form of the current carrying region 2801 according to the fifth example in more detail.

In the fifth example, the horizontal length of the row contact unit 1401 and the horizontal length of the column contact unit 1402 may be the same, and the vertical length of the row contact unit 1401 and the vertical length of the column contact unit 1402 may be the same.

The current carrying region 2701 according to an embodiment of the present invention is formed to correspond to at least one key region 1701.

When viewing the form of the current carrying region of the insulating layer 1801 in the fifth example together with FIG. 23, the current carrying region of the insulating layer 1801 has a form in which the line-crossing region 2301 is insulated while allowing the current to be carried in the point-crossing region 2302. As illustrated, in the current carrying region of the insulating layer 1801 according to an embodiment of the present invention, the current may be allowed to be carried in the region where the line-crossing region 2301 and the point-crossing region 2302 overlap.

To this end, the insulating layer 1801 according to an embodiment of the present invention, the insulating layer 1801 forms the current carrying region 2801 in a rectangular shape corresponding to the key region 1701, but may further include at least one protruding insulating part 2901-1 to 2901-8 for covering the line-crossing region 2301. That is, the at least one protruding insulating portion 2901-1 to 2901-8 is provided in a shape protruding from the edge of the rectangular current carrying region 2801 toward the inside, and cover the line-crossing region 2301.

That is, in the insulating region of the insulating layer 1801 according to an embodiment of the present invention, at least one protruding insulating part 2901-1 to 2901-8 provided in the shape protruding from the edge of the current carrying region toward the inside may be formed.

In the illustrated example, the at least one protruding insulating portion 2901-1 to 2901-8 is illustrated in a rectangular shape protruding from the edge toward the inside, but it will not necessarily be limited to this shape.

The protrusion height (d, 2902) of the at least one protruding insulating part 2901-1 to 2901-8 according to an embodiment of the present invention is formed to cover the outermost electrode line among the electrode line bundles 1502 arranged at the edge, and may be formed so as not to cover the second or more electrode lines among the electrode line bundles 1502 disposed at the edge.

That is, the protrusion height of the at least one protruding insulating part 2901-1 to 2901-8 according to an embodiment of the present invention may be formed to cover the outermost electrode line.

The effects of the key input device, the smart mat based on the key input device, the control method thereof according to the present disclosure will be described as follows.

According to at least one of the embodiments of the present disclosure, there is an advantage in that a user's touch or pressure can be detected sensed without using the key having the physical form.

In addition, according to at least one of the embodiments of the present disclosure, there is an advantage in that a key input made on a plurality of points on a large region such as a mat can be more accurately detected

Although the embodiment of the key input device, the smart mat based on the key input device, and the control method thereof according to the present disclosure has been described above, it is described as at least one embodiment, and the technical spirit of the present invention and its configuration and operation are not limited by the described embodiment, and the scope of the technical spirit of the present invention is not restricted/limited by the drawings or the description made with reference to the drawings. In addition, the concepts and embodiments of the invention presented in the present invention can be used by those of ordinary skill in the art to which the present invention belongs as a basis for modifying or designing the structure of the present invention to other structures in order to perform the same purpose of the present invention, and equivalent structures obtained by modifying or changing the structure of the present invention by those of ordinary skill in the art to which the present invention belongs are bound by the technical scope of the present invention set forth in the claims, and various alterations, substitutions, and changes can be made thereto without departing from the spirit or scope of the invention described in the claims.

Although the key input device, smart mat including key input device, interactive fitness system and control method thereof have been described with reference to the specific embodiments, they are not limited thereto. Therefore, it will be readily understood by those skilled in the art that various modifications and changes can be made thereto without departing from the spirit and scope of the present invention defined by the appended claims.

What is claimed is:

1. A key input device for a smart mat, comprising:
a first layer in which a plurality of row contacts are formed;
a second layer in which a plurality of column contacts are formed;
an insulating layer disposed between the first and second layers to form an insulating region and a current carrying region; and
a processor configured to detect that at least one of a plurality of key switches formed by the plurality of row contacts and the plurality of column contacts is pressed.

2. The key input device of claim 1, wherein a key region is formed in a region where the plurality of row and column contacts cross each other.

3. The key input device of claim 2, wherein the current carrying region is formed to correspond to the key region.

4. The key input device of claim 3, wherein:
the plurality of row contacts include a plurality of row contact units connected to each other, and
the plurality of column contacts include a plurality of column contact units connected to each other.

5. The key input device of claim 4, wherein the key region is formed at a point where the plurality of row contact units and column contact units cross each other.

6. The key input device of claim 5, wherein the insulating region is formed to insulate a region where the row contact unit and the column contact unit cross along a line.

7. The key input device of claim 6, wherein the current carrying region is formed such that the current is allowed to be carried in a region where the row contact unit and the column contact unit cross each other at a point.
